(19) United States
(12) Patent Application Publication
(10) Pub. No.: US 2022/0185857 A1
(43) Pub. Date: Jun. 16, 2022

(54) FMS-LIKE TYROSINE KINASE 3 LIGAND (FLT3L)-BASED CHIMERIC PROTEINS

(71) Applicants: Orionis Biosciences, Inc., Waltham, MA (US); VIB VZW, Gent (BE); Universiteit Gent, Gent (BE)

(72) Inventors: Nikolai KLEY, Waltham, MA (US); Jan TAVERNIER, Balegem (BE); Alexander VAN PARYS, Gent (BE)

(21) Appl. No.: 17/442,772

(22) PCT Filed: Mar. 27, 2020
(86) PCT No.: PCT/US20/25419

§ 371 Date: Sep. 24, 2021

Related U.S. Application Data
(60) Provisional application No. 62/825,579, filed on Mar. 28, 2019.

(51) Int. Cl. C07K 14/52; C07K 14/56; C07K 14/565; C07K 14/545

(52) U.S. Cl. CPC C07K 14/52; C07K 14/56; A61K 38/00; C07K 14/545; C07K 2319/02; C07K 14/565

(57) ABSTRACT

FMS-like tyrosine kinase 3L (FLT3L) fused to human cytokines, which find use in, e.g., cancer treatments, is described.

### CROSS-REFERENCE TO RELATED APPLICATIONS

This application claims the benefit of and priority to U.S. Provisional Patent Application No. 62/825,579 filed Mar. 28, 2019, the contents of which are hereby incorporated by reference in their entirety.

### FIELD

FMS-like tyrosine kinase 3 ligand (FLT3L) fused to a signaling agent, for instance, without limitation, human IFNα2, IFNβ, and IL-1β, which finds use in, e.g. cancer treatments, is described.

### SEQUENCE LISTING

The instant application contains a Sequence Listing that has been submitted in ASCII format via EFS-Web and is hereby incorporated by reference in its entirety. Said ASCII copy, created on Mar. 23, 2020, is named ORN-062PC_A_Sequence_Listing_ST25.txt and is 28,672 bytes in size.

### BACKGROUND

FMS-like tyrosine kinase 3 (FLT3) is expressed on the surface of many hematopoietic progenitor cells. Signaling of FLT3 is important for the normal development of hematopoietic stem cells and progenitor cells. The FLT3 gene is one of the most frequently mutated genes in acute myeloid leukemia (AML). Further, FMS-like tyrosine kinase 3 ligand (FLT3L) agents find use in priming the immune system, e.g., altering the number of dendritic cells. Cytokines are naturally occurring substances capable of modulating cellular growth and differentiation. Cytokines play important roles in a variety of physiological processes including, for example, metabolism, respiration, sleep, excretion, healing, movement, reproduction, mood, stress, tissue function, immune function, sensory perception, and growth and development.

Clinically, cytokines would seem to be applicable to the treatment of a variety of diseases and disorders including, for example, cancers. However, the administration of these soluble agents is not without risks. The therapeutic use of cytokines is often associated with systemic toxicity and deleterious side effects thus limiting the dose levels that these agents can be used.

### SUMMARY OF THE INVENTION

Accordingly, in some aspects, the present invention relates to a chimeric protein comprising a targeting moiety which comprises a single copy of FMS-like tyrosine kinase 3 ligand (FLT3L), or a portion thereof. In various embodiments, the targeting moiety functionally modulates the antigen or receptor of interest. In some embodiments, the targeting moiety binds but does not functionally modulate the antigen or receptor of interest. In some embodiments, the targeting moiety comprises a single copy of the extracellular domain of FLT3L, or respective portions thereof. The chimeric protein in accordance with embodiments of the present invention also comprises a signaling agent or a modified form thereof, the signaling agent as described herein, for instance, without limitation, human IFNα2, IFNβ, and IL-1β. The chimeric protein also comprises one or more flexible linkers connecting the chimeric protein and the signaling agent.

In some embodiments, the signaling agent can be a wild type signaling agent as described herein, for instance, without limitation, human IFNα2, IFNβ, and IL-1β. In other embodiments, the signaling agent can be modified to comprise one or more mutations. The one or more mutations introduced into the signaling agent can confer various improved properties upon the chimeric protein compared to a chimeric protein with an unmodified (e.g., wild type) signaling agent. For example, the signaling agent can be a mutant human signaling agent as described herein, for instance, without limitation, human IFNα2, IFNβ, and IL-1β, having one or more mutations that confer improved safety as compared to the wild type signaling agent as described herein, for instance, without limitation, human IFNα2, IFNβ, and IL-1β. In various embodiments, the one or more mutations can confer improved safety as compared to a wild type signaling agent, reduced affinity for the signaling agent's receptor, or reduced bioactivity for the signaling agent's receptor. In some embodiments, the one or more mutations allow for attenuation of the signaling agent's activity; for example, agonistic or antagonistic activity of the signaling agent may be attenuated. In some embodiments, one or more mutations of the modified signaling agent convert the signaling agent's activity from agonistic to antagonistic. In various embodiments, the mutation(s) confer reduced affinity or activity that is restorable by attachment to one or more targeting moiety. Further, in various embodiments, the mutation(s) confer substantially reduced or ablated affinity or activity that is not substantially restorable by attachment to a targeting moiety.

In various embodiments, the targeting moiety is directed against an immune cell, such that it directly or indirectly recruits immune cells to tumor cells or to the tumor microenvironment. Non-limiting examples of an immune cell include a dendritic cell, a T cell, a B cell, a macrophage, a neutrophil, myeloid derived suppressor cell, or a NK cell. In some embodiments, targeting moiety is directed to a hematopoietic stem cell (HSC), early progenitor cell, immature thymocyte, or steady state dendritic cell (DC). In embodiments, the targeting is to a dendritic cell, such as a conventional dendritic cell (cDC) or plasmacytoid dendritic cells (pDC). In embodiments, the targeting is to a cDC, optionally being cDC-1, migratory DCs, and Flt3+ DCs. In some embodiments, the targeting moiety may increase a number of dendritic cells. In some embodiments, the targeting moiety of the present chimeric proteins enhances tumor antigen presentation, optionally by dendritic cells.

In various embodiments, the present chimeric proteins find use in a patient having various diseases or disorders such as one or more of cancer, infections, immune disorders, autoimmune and/or neurodegenerative disease, cardiovascular diseases, wound, ischemia-related diseases, metabolic diseases, and/or many other diseases and disorders. The present invention encompasses various methods of treating or preventing diseases and disorders, for instance, various type of cancer and an autoimmune and/or neurodegenerative disease. In some embodiments, the cancer is acute myeloid leukemia (AML).

### BRIEF DESCRIPTION OF THE DRAWINGS

FIG. 1 shows tumor growth curves in humanized mice after treatment with buffer or Flt3L-AcTaferon (i.e. a chimera of the Flt3L ECD with IFNα2, R149A mutant). Average values (in mm³) of 5 or 6 animals per time point time (+SEM) are plotted.

FIG. 2 is a SEC (size exclusion chromatography) profile of purified FLT3L-AFN (dark line) with protein markers shown in grey line.

FIG. 3 is a SDS-PAGE gel under non-reducing conditions of fractions 2, 3, 4 of the SEC column.

### DETAILED DESCRIPTION

In some aspects, a chimeric protein is provided that comprises a targeting moiety which comprises a single copy of FMS-like tyrosine kinase 3 ligand (FLT3L), or a portion thereof. The chimeric protein also comprises a wild type signaling agent or a modified form thereof, the signaling agent signaling being one of those described herein, for instance, without limitation, human IFNα2, IFNβ, and IL-1β, which, in various embodiments, can be wild type human or a mutant form. In the chimeric protein, one or more flexible linkers connect the targeting moiety and the signaling agent.

In some embodiments, the targeting moiety comprises a single copy of a portion of FLT3L. In other embodiments, the targeting moiety comprises a single copy of the extracellular domain of FLT3L, or a portion thereof. In some embodiments, the targeting moiety comprises an amino acid sequence which is a truncation of SEQ ID NO: 1. The amino acid sequence for SEQ ID NO: 1 (F1t3L full length) is:

MTVLAPAWSPTTYLLLLLLLSSGLSGTQDCSFQHSPISSDFAVKIRELSD
YLLQDYPVTVASNLQDEELCGGLWRLVLAQRWMERLKTVAGSKMQGLLER
VNTEIHFVTKCAFQPPPSCLRFVQTNISRLLQETSEQLVALKPWITRQNF
SRCLELQCQPDSSTLPPPWSPRPLEATAPTAPQPPLLLLLLLPVGLLLLA
AAWCLHWQRTRRRTPRPGEQVPPVPSPQDLLLVEH

where Bold=leader sequence, Underlined: extracellular region not part of receptor binding domain, Italic=transmembrane and intracellular domain.

In some embodiments, the targeting moiety comprises an amino acid sequence having at least 90% identity with any one of SEQ ID NOs: 2-5, or an amino acid sequence having at least 95% identity with any one of SEQ ID NOs: 2-5.

In some embodiments, the targeting moiety comprises a single copy of an amino acid sequence having at least 90% identity with any one of SEQ ID NOs: 2-5, or an amino acid sequence having at least 95% identity with any one of SEQ ID NOs: 2-5.

The amino acid sequence for SEQ ID NO: 2 (mature Flt3L-ec (extracellular domain)) is:

TQDCSFQHSPISSDFAVKIRELSDYLLQDYPVTVASNLQDEELCGGLWRL
VLAQRWMERLKTVAGSKMQGLLERVNTEIHFVTKCAFQPPPSCLRFVQTN
ISRLLQETSEQLVALKPWITRQNFSRCLELQCQPDSSTLPPPWSPRPLEA
TAPTAPQP.

The amino acid sequence for SEQ ID NO: 3 (mature Flt3L-ec (extracellular domain) function shorter variant commercial source (Prospecbio)) is:

TQDCSFQHSPISSDFAVKIRELSDYLLQDYPVTVASNLQDEELCGGLWRL
VLAQRWMERLKTVAGSKMQGLLERVNTEIHFVTKCAFQPPPSCLRFVQTN
ISRLLQETSEQLVALKPWITRQNFSRCLELQCQPDSSTLPPPWSPRPLEA
TAPTA

The amino acid sequence for SEQ ID NO: 4 mature Flt3L-ec (extracellular domain) minimal functional domain (Savvides et al., 2000, Nature Structural Biology) is:

TQDCSFQHSPISSDFAVKIRELSDYLLQDYPVTVASNLQDEELCGGLWRL
VLAQRWMERLKTVAGSKMQGLLERVNTEIHFVTKCAFQPPPSCLRFVQTN
ISRLLQETSEQLVALKPWITRQNFSRCLELQCQP

The amino acid sequence for SEQ ID NO: 5 mature Flt3L-ec (extracellular domain) minimal functional domain (Savvides et al., 2000, Nature Structural Biology) shortened by starting at the first cysteine and ending at the last cysteine is:

CSFQHSPISSDFAVKIRELSDYLLQDYPVTVASNLQDEELCGGLWRLVLA
QRWMERLKTVAGSKMQGLLERVNTEIHFVTKCAFQPPPSCLRFVQTNISR
LLQETSEQLVALKPWITRQNFSRCLELQC

In some embodiments, the chimeric protein of the present invention is a dimer. In embodiments, the chimeric protein is a non-covalently linked dimer. In some embodiments, the chimeric protein of the present invention comprises the amino acid sequence of SEQ ID NO: 9, or a variant having at least about 90%, 95%, 97%, 98%, or 99% identity thereto.

In some embodiments, the signaling agent comprises an amino acid sequence having at least 95% identity with one of SEQ ID NO: 6, 7, 38, or 39, or the signaling agent can comprise an amino acid sequence of one of SEQ ID NO: 6, 7, 38, or 39.

In various embodiments, the signaling agent is a modified (e.g., mutant) form of the signaling agent having one or more mutations. In various embodiments, the mutations allow for the modified signaling agent to have one or more of attenuated activity such as one or more of reduced binding affinity, reduced endogenous activity, and reduced specific bioactivity relative to unmodified or unmutated, i.e. the wild type form of the signaling agent (e.g. comparing the same signaling agent in a wild type form versus a modified (e.g. mutant) form). In various embodiments, the mutations allow for the modified signaling agent to have one or more of attenuated activity such as one or more of reduced binding affinity, reduced endogenous activity, and reduced specific bioactivity relative to unmodified or unmutated, e.g. wild type IFNα2, IFNβ, or IL-1β. In some embodiments, the mutations that attenuate or reduce binding or affinity include those mutations that substantially reduce or ablate binding or activity. In some embodiments, the mutations that attenuate or reduce binding or affinity are different than those mutations which substantially reduce or ablate binding or activity. Consequentially, in various embodiments, the mutations allow for the signaling agent to be more safe, e.g. have reduced systemic toxicity, reduced side effects, and reduced off-target effects relative to unmutated, i.e. wild type, signaling agent (e.g. comparing the same signaling agent in a wild type form versus a modified (e.g. mutant) form). In various embodiments, the mutations allow for the signaling agent to be safer, e.g. have reduced systemic toxicity, reduced side effects, and reduced off-target effects relative to unmutated interferon, e.g. the unmutated sequence of IFNα2, IFNβ, or IL-1β.

In various embodiments, the signaling agent is modified to have one or more mutations that reduce its binding affinity or activity for one or more of its receptors. In some embodiments, the signaling agent is modified to have one or more mutations that substantially reduce or ablate binding affinity or activity for the receptors. In some embodiments, the activity provided by the wild type signaling agent is agonism at the receptor (e.g. activation of a cellular effect at a site of therapy). For example, the wild type signaling agent may activate its receptor. In such embodiments, the mutations result in the modified signaling agent to have reduced or ablated activating activity at the receptor. For example, the mutations may result in the modified signaling agent to deliver a reduced activating signal to a target cell or the activating signal could be ablated. In some embodiments, the activity provided by the wild type signaling agent is antagonism at the receptor (e.g. blocking or dampening of a cellular effect at a site of therapy). For example, the wild type signaling agent may antagonize or inhibit the receptor. In these embodiments, the mutations result in the modified signaling agent to have a reduced or ablated antagonizing activity at the receptor. For example, the mutations may result in the modified signaling agent to deliver a reduced inhibitory signal to a target cell or the inhibitory signal could be ablated. In various embodiments, the signaling agent is antagonistic due to one or more mutations, e.g. an agonistic signaling agent is converted to an antagonistic signaling agent (e.g. as described in WO 2015/007520, the entire contents of which are hereby incorporated by reference) and, such a converted signaling agent, optionally, also bears one or more mutations that reduce its binding affinity or activity for one or more of its receptors or that substantially reduce or ablate binding affinity or activity for one or more of its receptors.

In some embodiments, the reduced affinity or activity at the receptor is restorable by attachment with one or more of the targeting moieties. In other embodiments, the reduced affinity or activity at the receptor is not substantially restorable by the activity of one or more of the targeting moieties.

In various embodiments, the signaling agent is active on target cells because the targeting moiety compensates for the missing/insufficient binding (e.g., without limitation and/or avidity) required for substantial activation. In various embodiments, the modified signaling agent is substantially inactive en route to the site of therapeutic activity and has its effect substantially on specifically targeted cell types that greatly reduces undesired side effects. In some embodiments, the signaling agent may include one or more mutations that attenuate or reduce binding or affinity for one receptor (i.e., a therapeutic receptor) and one or more mutations that substantially reduce or ablate binding or activity at a second receptor. In such embodiments, these mutations may be at the same or at different positions (i.e., the same mutation or multiple mutations). In some embodiments, the mutation(s) that reduce binding and/or activity at one receptor is different than the mutation(s) that substantially reduce or ablate at another receptor. In some embodiments, the mutation(s) that reduce binding and/or activity at one receptor is the same as the mutation(s) that substantially reduce or ablate at another receptor. In some embodiments, the present chimeric proteins have a modified signaling agent that has both mutations that attenuate binding and/or activity at a therapeutic receptor and therefore allow for a more controlled, on-target therapeutic effect (e.g. relative wild type signaling agent) and mutations that substantially reduce or ablate binding and/or activity at another receptor and therefore reduce side effects (e.g. relative to wild type signaling agent).

In some embodiments, the substantial reduction or ablation of binding or activity is not substantially restorable with a targeting moiety. In some embodiments, the substantial reduction or ablation of binding or activity is restorable with a targeting moiety. In various embodiments, substantially reducing or ablating binding or activity at a second receptor also may prevent deleterious effects that are mediated by the other receptor. Alternatively, or in addition, substantially reducing or ablating binding or activity at the other receptor causes the therapeutic effect to improve as there is a reduced or eliminated sequestration of the therapeutic chimeric proteins away from the site of therapeutic action. For instance, in some embodiments, this obviates the need of high doses of the present chimeric proteins that compensate for loss at the other receptor. Such ability to reduce dose further provides a lower likelihood of side effects.

In various embodiments, the modified signaling agent comprises one or more mutations that cause the signaling agent to have reduced, substantially reduced, or ablated affinity, e.g. binding (e.g. K_{D}) and/or activation (for instance, when the modified signaling agent is an agonist of its receptor, measurable as, for example, K_{A} and/or EC₅₀) and/or inhibition (for instance, when the modified signaling agent is an antagonist of its receptor, measurable as, for example, Kₗ and/or 10₅₀), for one or more of its receptors. In various embodiments, the reduced affinity at the signaling agent's receptor allows for attenuation of activity (inclusive of agonism or antagonism). In such embodiments, the modified signaling agent has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity for the receptor relative to the wild type signaling agent. In some embodiments, the binding affinity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower relative to the wild type signaling agent (including, by way of non-limitation, relative to the unmutated IFNα2, IFNβ, or IL-1β).

In embodiments wherein the chimeric protein has mutations that reduce binding at one receptor and substantially reduce or ablate binding at a second receptor, the attenuation or reduction in binding affinity of a modified signaling agent for one receptor is less than the substantial reduction or ablation in affinity for the other receptor. In some embodiments, the attenuation or reduction in binding affinity of a modified signaling agent for one receptor is less than the substantial reduction or ablation in affinity for the other receptor by about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, or about 95%. In various embodiments, substantial reduction or ablation refers to a greater reduction in binding affinity and/or activity than attenuation or reduction.

In various embodiments, the modified signaling agent comprises one or more mutations that reduce the endogenous activity of the signaling agent to about 75%, or about 70%, or about 60%, or about 50%, or about 40%, or about 30%, or about 25%, or about 20%, or about 10%, or about 5%, or about 3%, or about 1%, e.g., relative to the wild type signaling agent (including, by way of non-limitation, relative to the unmutated IFNα2, IFNβ, or IL-1β).

In various embodiments, the modified signaling agent comprises one or more mutations that cause the signaling agent to have reduced affinity and/or activity for a receptor of any one of the cytokines, growth factors, and hormones as described herein.

In some embodiments, the modified signaling agent comprises one or more mutations that cause the signaling agent to have reduced affinity for its receptor that is lower than the binding affinity of the targeting moiety for its(their) receptor(s). In some embodiments, this binding affinity differential is between signaling agent/receptor and targeting moiety/receptor on the same cell. In some embodiments, this binding affinity differential allows for the signaling agent, e.g. mutated signaling agent, to have localized, on-target effects and to minimize off-target effects that underlie side effects that are observed with wild type signaling agent. In some embodiments, this binding affinity is at least about 2-fold, or at least about 5-fold, or at least about 10-fold, or at least about 15-fold lower, or at least about 25-fold, or at least about 50-fold lower, or at least about 100-fold, or at least about 150-fold. Receptor binding activity may be measured using methods known in the art. For example, affinity and/or binding activity may be assessed by Scatchard plot analysis and computer-fitting of binding data (e.g. Scatchard, 1949 Annals of the New York Academy of Sciences. 51 (4): 660-672) or by reflectometric interference spectroscopy under flow through conditions, as described by Brecht et al. (1993), Biosens Bioelectron 1993;8:387-392 the entire contents of all of which are hereby incorporated by reference.

In embodiments, the wild type or modified signaling agent is an interferon is a type I interferon. In embodiments, the wild type or modified signaling agent is selected from IFNα2, IFN-α1, IFN-β, IFN-γ, Consensus IFN, IFN-_{ε}, IFN-_{K}, IFN-_{T}, IFN-δ, and IFN-v.

In embodiments, the wild type or modified signaling agent is interferon α. In such embodiments, the modified IFNα2 agent has reduced affinity and/or activity for the IFN-α/β receptor (IFNAR), i.e., IFNAR1 and/or IFNAR2 chains. In some embodiments, the modified IFNα2 agent has substantially reduced or ablated affinity and/or activity for the IFN-α/β receptor (IFNAR), i.e., IFNAR1 and/or IFNAR2 chains.

Mutant forms of interferon α2 are known to the person skilled in the art. In an illustrative embodiment, the modified signaling agent is the allelic form IFNα2a having the amino acid sequence of:

(SEQ ID NO: 6)
CDLPQTHSLGSRRTLMLLAQMRKISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKE.

In an illustrative embodiment, the wild type or modified signaling agent is the allelic form IFNα2b having the amino acid sequence of:

CDLPQTHSLGSRRTLMLLAQMRRISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEWRAEIMRSF
SLSTNLQESLRSKE
(SEQ ID NO: 7, which differs from IFNα2a at amino
acid position 23).

In some embodiments, said IFNα2 mutant (IFNα2a or IFNα2b) is mutated at one or more amino acids at positions 144-154, such as amino acid positions 148, 149 and/or 153. In some embodiments, the IFNα2 mutant comprises one or more mutations selected from L153A, R149A, and M148A. Such mutants are described, for example, in WO2013/107791 and Piehler et al., (2000) J. Biol. Chem, 275:40425-33, the entire contents of all of which are hereby incorporated by reference.

In some embodiments, the IFNα2 mutants have reduced affinity and/or activity for IFNAR1. In some embodiments, the IFNα2 mutant comprises one or more mutations selected from F64A, N65A, T69A, L80A, Y85A, and Y89A, as described in WO2010/030671, the entire contents of which is hereby incorporated by reference.

In some embodiments, the IFNα2 mutant comprises one or more mutations selected from K133A, R144A, R149A, and L153A as described in WO2008/124086, the entire contents of which is hereby incorporated by reference.

In some embodiments, the IFNα2 mutant comprises one or more mutations selected from R120E and R120E/K121E, as described in WO2015/007520 and WO2010/030671, the entire contents of which are hereby incorporated by reference. In such embodiments, said IFNα2 mutant antagonizes wildtype IFNα2 activity. In such embodiments, said mutant IFNα2 has reduced affinity and/or activity for IFNAR1 while affinity and/or activity of IFNR2 is retained.

In some embodiments, the human IFNα2 mutant comprises (1) one or more mutations selected from R120E and R120E/K121E, which, without wishing to be bound by theory, create an antagonistic effect and (2) one or more mutations selected from K133A, R144A, R149A, and L153A, which, without wishing to be bound by theory, allow for an attenuated effect at, for example, IFNAR2. In an embodiment, the human IFNα2 mutant comprises R120E and L153A.

In some embodiments, the human IFNα2 mutant comprises one or more mutations selected from, L15A, A19W, R22A, R23A, L26A, F27A, L30A, L30V, K31A, D32A, R33K, R33A, R33Q, H34A, D35A, Q40A, D114R, L117A, R120A, R125A, K134A, R144A, A145G, A145M, M148A, R149A, S152A, L153A, and N156A as disclosed in WO 2013/059885, the entire disclosures of which are hereby incorporated by reference. In some embodiments, the human IFNα2 mutant comprises the mutations H57Y, E58N, Q61S, and/or L30A as disclosed in WO 2013/059885. In some embodiments, the human IFNα2 mutant comprises the mutations H57Y, E58N, Q61S, and/or R33A as disclosed in WO 2013/059885. In some embodiments, the human IFNα2 mutant comprises the mutations H57Y, E58N, Q61S, and/or M148A as disclosed in WO 2013/059885. In some embodiments, the human IFNα2 mutant comprises the mutations H57Y, E58N, Q61S, and/or L153A as disclosed in WO 2013/059885. In some embodiments, the human IFNα2 mutant comprises the mutations N65A, L80A, Y85A, and/or Y89A as disclosed in WO 2013/059885. In some embodiments, the human IFNα2 mutant comprises the mutations N65A, L80A, Y85A, Y89A, and/or D114A as disclosed in WO 2013/059885.

In various embodiments, the signaling agent is a mutant human IFNα2. In some embodiments, the mutant human IFNα2 comprises an amino acid sequence having at least 95% identity with SEQ ID NO: 6 or 7, wherein the mutant human IFNα2 has one or more mutations that confer improved safety as compared to a wild type IFNα2 having an amino acid sequence of SEQ ID NO: 6 or 7. In some embodiments, the IFNα2 has one or more mutations at positions 144 to 154 with respect to SEQ ID NO: 6 or 7. In some embodiments, the human IFNα2 has one or more mutations at positions L15, A19, R22, R23, L26, F27, L30, L30, K31, D32, R33, H34, D35, Q40, H57, E58, Q61, F64, N65, T69, L80, Y85, Y89, D114, L117, R120, R125, K133, K134, R144, A145, M148, R149, S152, L153, and N156 with respect to SEQ ID NO: 6 or 7. In some embodiments, the mutant IFNα2 has one or more mutations at position R149, M148, or L153 with respect to SEQ ID NO: 6 or 7. In some embodiments, the one or more mutations are one or more of L15A, A19W, R22A, R23A, L26A, F27A, L30A, L30V, K31A, D32A, R33K, R33A, R33Q, H34A, D35A, Q40A, H57Y, E58N, Q61S, F64A, N65A, T69A, L80A, Y85A, Y89A, D114R, L117A, R120A, R125A, K133A, K134A, R144A, A145G, A145M, M148A, R149A, S152A, L153A, and N156A with respect to SEQ ID NO: 6 or 7.In some embodiments, the mutant human IFNα2 has R149A mutation with respect to SEQ ID NO: 6 or 7.

In some embodiments, the mutant human IFNα2 has one or more mutations at position R33, R144, A145, M148, R149, and L153 with respect to SEQ ID NO: 6 or 7. In some embodiments, the mutant human IFNα2 has a R33A, R144A, R144I, R144L, R144S, R144T, R144Y, A145D, A145G, A145H, A145K, A145Y, M148A, R149A, and L153A mutation with respect to SEQ ID NO: 6 or 7.

In some embodiments, the mutant human IFNα2 has one or more mutations at position R33, T106, R144, A145, M148, R149, and L153 with respect to SEQ ID NO: 6 or 7. In some embodiments, the mutant human IFNα2 has one or more mutations selected from R33A, T106X₃, R120E, R144X₁ A145X₂, M148A, R149A, and L153A with respect to amino acid sequence of SEQ ID NO: 6 or 7, wherein X₁ is selected from A, S, T, Y, L, and I, wherein X2 is selected from G, H, Y, K, and D, and wherein X3 is selected from A and E

In embodiments, the wild type or modified signaling agent is IFN-β. In some embodiments, the IFN-β is human having a sequence as shown below:

(SEQ ID NO: 38)
MSYNLLGFLQRSSNFQCQKLLWQLNGRLEYCLKDRMNFDIPEEIKQLQQF
QKEDAALTIYEMLQNIFAIFRQDSSSTGWNETIVENLLANVYHQINHLKT
VLEEKLEKEDFTRGKLMSSLHLKRYYGRILHYLKAKEYSHCAWTIVRVEI
LRNFYFINRLTGYLRN

In various embodiments, the IFN-β encompasses functional derivatives, analogs, precursors, isoforms, splice variants, or fragments of IFN-β. In various embodiments, the IFN-β encompasses IFN-β derived from any species. In an embodiment, chimeric protein comprises a modified version of mouse IFN-β. In another embodiment, the chimeric protein comprises a modified version of human IFN-β. Human IFN-β is a polypeptide with a molecular weight of about 22 kDa comprising 166 amino acid residues. The amino acid sequence of human IFN-β is SEQ ID NO: 38.

In some embodiments, the human IFN-β is IFN-β-1a which is a glycosylated form of human IFN-β. In some embodiments, the human IFN-β is IFN-β-1b which is a non-glycosylated form of human IFN-β that has a Met-1 deletion and a Cys-17 to Ser mutation.

In various embodiments, the modified IFN-β has one or more mutations that reduce its binding to or its affinity for the IFNAR1 subunit of IFNAR. In one embodiment, the modified IFN-β has reduced affinity and/or activity at IFNAR1. In various embodiments, the modified IFN-β is human IFN-β and has one or more mutations at positions F67, R71, L88, Y92, I95, N96, K123, and R124. In some embodiments, the one or more mutations are substitutions selected from F67G, F67S, R71A, L88G, L885, Y92G, Y92S, I95A, N96G, K123G, and R124G. In an embodiment, the modified IFN-β comprises the F67G mutation. In an embodiment, the modified IFN-β comprises the K123G mutation. In an embodiment, the modified IFN-β comprises the F67G and R71A mutations. In an embodiment, the modified IFN-β comprises the L88G and Y92G mutations. In an embodiment, the modified IFN-β comprises the Y92G, I95A, and N96G mutations. In an embodiment, the modified IFN-β comprises the K123G and R124G mutations. In an embodiment, the modified IFN-β comprises the F67G, L88G, and Y92G mutations. In an embodiment, the modified IFN-β comprises the F67S, L885, and Y92S mutations.

In some embodiments, the modified IFN-β has one or more mutations that reduce its binding to or its affinity for the IFNAR2 subunit of IFNAR. In one embodiment, the modified IFN-β has reduced affinity and/or activity at IFNAR2. In various embodiments, the modified IFN-β is human IFN-β and has one or more mutations at positions W22, R27, L32, R35, V148, L151, R152, and Y155. In some embodiments, the one or more mutations are substitutions selected from W22G, R27G, L32A, L32G, R35A, R35G, V148G, L151G, R152A, R152G, and Y155G. In an embodiment, the modified IFN-β comprises the W22G mutation. In an embodiment, the modified IFN-β comprises the L32A mutation. In an embodiment, the modified IFN-β comprises the L32G mutation. In an embodiment, the modified IFN-β comprises the R35A mutation. In an embodiment, the modified IFN-β comprises the R35G mutation. In an embodiment, the modified IFN-β comprises the V148G mutation. In an embodiment, the modified IFN-β comprises the R152A mutation. In an embodiment, the modified IFN-β comprises the R152G mutation. In an embodiment, the modified IFN-β comprises the Y155G mutation. In an embodiment, the modified IFN-β comprises the W22G and R27G mutations. In an embodiment, the modified IFN-β comprises the L32A and R35A mutation. In an embodiment, the modified IFN-β comprises the L151G and R152A mutations. In an embodiment, the modified IFN-β comprises the V148G and R152A mutations.

In some embodiments, the modified IFN-β has one or more of the following mutations: R35A, R35T, E42K, M62l, G78S, A141Y, A142T, E149K, and R152H. In some embodiments, the modified IFN-β has one or more of the following mutations: R35A, R35T, E42K, M62I, G78S, A141Y, A142T, E149K, and R152H in combination with C17S or C17A.

In some embodiments, the modified IFN-β has one or more of the following mutations: R35A, R35T, E42K, M62l, G78S, A141Y, A142T, E149K, and R152H in combination with any of the other IFN-β mutations described herein.

The crystal structure of human IFN-β is known and is described in Karpusas et al., (1998) PNAS, 94(22): 11813-11818. Specifically, the structure of human IFN-β has been shown to include five α-helices (i.e., A, B, C, D, and E) and four loop regions that connect these helices (i.e., AB, BC, CD, and DE loops). In various embodiments, the modified IFN-β has one or more mutations in the A, B, C, D, E helices and/or the AB, BC, CD, and DE loops which reduce its binding affinity or activity at a therapeutic receptor such as IFNAR. Exemplary mutations are described in WO2000/023114 and US20150011732, the entire contents of which are hereby incorporated by reference. In an exemplary embodiment, the modified IFN-β is human IFN-β comprising alanine substitutions at amino acid positions 15, 16, 18, 19, 22, and/or 23. In an exemplary embodiment, the modified IFN-β is human IFN-β comprising alanine substitutions at amino acid positions 28-30, 32, and 33. In an exemplary embodiment, the modified IFN-β is human IFN-β comprising alanine substitutions at amino acid positions 36, 37, 39, and 42. In an exemplary embodiment, the modified IFN-β is human IFN-β comprising alanine substitutions at amino acid positions 64 and 67 and a serine substitution at position 68. In an exemplary embodiment, the modified IFN-β is human IFN-β comprising alanine substitutions at amino acid positions 71-73. In an exemplary embodiment, the modified IFN-β is human IFN-β comprising alanine substitutions at amino acid positions 92, 96, 99, and 100. In an exemplary embodiment, the modified IFN-β is human IFN-β comprising alanine substitutions at amino acid positions 128, 130, 131, and 134. In an exemplary embodiment, the modified IFN-β is human IFN-β comprising alanine substitutions at amino acid positions 149, 153, 156, and 159.

In some embodiments, the mutant IFNβ comprises SEQ ID NO: 38 and a mutation at W22, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at W22, the mutation being an aliphatic hydrophobic residue selected from glycine (G), alanine (A), leucine (L), isoleucine (I), methionine (M), and valine (V) and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L32, the mutation being an aliphatic hydrophobic residue selected from glycine (G), alanine (A), isoleucine (I), methionine (M), and valine (V).

In some embodiments, the mutant IFNβ comprises SEQ ID NO: 38 and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L32, the mutation being an aliphatic hydrophobic residue selected from glycine (G), alanine (A), isoleucine (I), methionine (M), and valine (V) and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at F67, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at F67, the mutation being an aliphatic hydrophobic residue selected from glycine (G), alanine (A), leucine (L), isoleucine (I), methionine (M), and valine (V) and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L88, the mutation being an aliphatic hydrophobic residue selected from glycine (G), alanine (A), isoleucine (I), methionine (M), and valine (V). In some embodiments, the mutant IFNβ comprises SEQ ID NO: 38 and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at F67, the mutation being an aliphatic hydrophobic residue selected from glycine (G), alanine (A), leucine (L), isoleucine (I), methionine (M), and valine (V) and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at I95, the mutation being an aliphatic hydrophobic residue selected from glycine (G), alanine (A), leucine (L), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at N96, the mutation being an aliphatic hydrophobic residue selected from glycine (G), alanine (A), leucine (L), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at Y92, the mutation being an aliphatic hydrophobic residue selected from glycine (G), alanine (A), leucine (L), isoleucine (I), methionine (M), and valine (V) and a mutation at I95, the mutation being an aliphatic hydrophobic residue selected from glycine (G), alanine (A), leucine (L), methionine (M), and valine (V) and a mutation at N96, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at K123, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at K123, the mutation being an aliphatic hydrophobic residue selected from glycine (G), alanine (A), leucine (L), isoleucine (I), methionine (M), and valine (V) and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L151, the mutation being an aliphatic hydrophobic residue selected from glycine (G), alanine (A), isoleucine (I), methionine (M), and valine (V). In some embodiments, the mutant IFNβ comprises SEQ ID NO: 38 and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L151, the mutation being an aliphatic hydrophobic residue selected from glycine (G), alanine (A), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at V148, the mutation being an aliphatic hydrophobic residue selected from glycine (G), alanine (A), leucine (L), isoleucine (I), and methionine (M). In some embodiments, the mutant IFNβ comprises SEQ ID NO: 38 and a mutation at V148, the mutation being an aliphatic hydrophobic residue selected from glycine (G), alanine (A), leucine (L), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at Y155, the mutation being an aliphatic hydrophobic residue selected from glycine (G), alanine (A), leucine (L), isoleucine (I), methionine (M), and valine (V).

In embodiments, the wild type or modified signaling agent is IL-1β. In an embodiment, the wild type IL-1β has the amino acid sequence of:

(SEQ ID NO: 39)
APVRSLNCTLRDSQQKSLVMSGPYELKALHLQGQDMEQQWFSMSFVQGEE
SNDKIPVALGLKEKNLYLSCVLKDDKPTLQLESVDPKNYPKKKMEKRFVF
NKIEINNKLEFESAQFPNWYISTSQAENMPVFLGGTKGGQDITDFTMQFV
SS.

IL-1β is a proinflammatory cytokine and an important immune system regulator. It is a potent activator of CD4 T cell responses, increases proportion of Th17 cells and expansion of IFNγ and IL-4 producing cells. IL-1β is also a potent regulator of CD8⁺ T cells, enhancing antigen-specific CD8⁺ T cell expansion, differentiation, migration to periphery and memory. IL-1β receptors comprise IL-1R1 and IL-1R2. Binding to and signaling through the IL-1R1 constitutes the mechanism whereby IL-1β mediates many of its biological (and pathological) activities. IL1-R2 can function as a decoy receptor, thereby reducing IL-1β availability for interaction and signaling through the IL-1R1.

In some embodiments, the wild type or modified signaling agent IL-1β has reduced affinity and/or activity (e.g. agonistic activity) for IL-1R1. In some embodiments, the modified IL-1β has substantially reduced or ablated affinity and/or activity for IL-1R2. In such embodiments, there is restorable IL-1β/IL-1R1 signaling and prevention of loss of therapeutic chimeric proteins at IL-R2 and therefore a reduction in dose of IL-1β that is required (e.g. relative to wild type or a chimeric protein bearing only an attenuation mutation for IL-R1). Such constructs find use in, for example, methods of treating cancer, including, for example, stimulating the immune system to mount an anti-cancer response.

In such embodiments, the modified signaling agent has a deletion of amino acids 52-54 which produces a modified human IL-1β with reduced binding affinity for type I IL-1R and reduced biological activity. See, for example, WO 1994/000491, the entire contents of which are hereby incorporated by reference. In some embodiments, the modified human IL-1β has one or more substitution mutations selected from A117G/P118G, R120X, L122A, T125G/L126G, R127G, Q130X, Q131G, K132A, S137G/Q138Y, L145G, H146X, L145A/L147A, Q148X, Q148G/Q150G, Q150G/D151A, M152G, F162A, F162A/Q164E, F166A, Q164E/E167K, N169G/D170G, I172A, V174A, K208E, K209X, K209A/K210A, K219X, E221X, E221 S/N224A, N224S/K225S, E244K, N245Q (where X can be any change in amino acid, e.g., a non-conservative change), which exhibit reduced binding to IL-1R, as described, for example, in WO2015/007542 and WO/2015/007536, the entire contents of which is hereby incorporated by reference (numbering base on the human IL-1β sequence, Genbank accession number NP_000567, version NP-000567.1 , GI: 10835145). In some embodiments, the modified human IL-1β may have one or more mutations selected from R120A, R120G, Q130A, Q130W, H146A, H146G, H146E, H146N, H146R, Q148E, Q148G, Q148L, K209A, K209D, K219S, K219Q, E221S and E221K. In an embodiment, the modified human IL-1β comprises the mutations Q131G and Q148G. In an embodiment, the modified human IL-1β comprises the mutations Q148G and K208E. In an embodiment, the modified human IL-1β comprises the mutations R120G and Q131G. In an embodiment, the modified human IL-1β comprises the mutations R120G and H146A. In an embodiment, the modified human IL-1β comprises the mutations R120G and H146N. In an embodiment, the modified human IL-1β comprises the mutations R120G and H146R. In an embodiment, the modified human IL-1β comprises the mutations R120G and H146E. In an embodiment, the modified human IL-1β comprises the mutations R120G and H146G. In an embodiment, the modified human IL-1β comprises the mutations R120G and K208E. In an embodiment, the modified human IL-1β comprises the mutations R120G, F162A, and Q164E. Modified human IL-1β mutations are relative to SEQ ID NO: 39.

In various embodiments, one or more mutations of the signaling agent may confer improved safety upon the chimeric protein as compared to a wild type signaling agent. The mutations may confer various other beneficial properties, including, without limitations, reduced affinity for the signaling agent's receptor and/or reduced bioactivity for the signaling agent's receptor. In some embodiments, the one or more mutations of the signaling agent allow for attenuation of the signaling agent's activity. For example, agonistic or antagonistic activity of the signaling agent can be attenuated. Furthermore, in some embodiments, the modified signaling agent comprises one or more mutations which convert its activity from agonistic to antagonistic.

In some embodiments, the signaling agent comprises one or more mutations that confer reduced affinity or activity that is restorable by attachment to one or more targeting moiety. In other embodiments, one or more mutations of the signaling agent confer substantially reduced or ablated affinity or activity that is not substantially restorable by attachment to a targeting moiety.

In some embodiments, the targeting moiety is directed against an immune cell, which can be selected from a dendritic cell, a T cell, a B cell, a macrophage, a neutrophil, myeloid derived suppressor cell, and a NK cell. In some embodiments, the targeting moiety is directed to a hematopoietic stem cell (HSC), early progenitor cell, immature thymocyte, or steady state dendritic cell (DC). The targeting moiety can functionally modulate the antigen or receptor of interest. In some embodiments, the targeting moiety binds but does not functionally modulate the antigen or receptor of interest.

In various embodiments, the chimeric protein, among other features, directly or indirectly recruits one or more immune cells to a disease cell, e.g. via the targeting moiety. Thus, in some embodiments, the targeting moiety directly or indirectly recruits immune cells to tumor cells or to the tumor microenvironment. In this way, the targeting moiety may increase a number of dendritic cells. In some embodiments, the targeting moiety enhances tumor antigen presentation, optionally by dendritic cells.

In various embodiments, the chimeric protein is suitable for use in a patient having one or more of cancer, infections, immune disorders, autoimmune and/or neurodegenerative disease, cardiovascular diseases, wound, ischemia-related diseases, and/or metabolic diseases. In some aspects, a method for treating or preventing a cancer is provided, that comprises administering an effective amount of the chimeric protein in accordance with various embodiments of the present disclosure to a patient in need thereof.

In various embodiments, the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome. In certain embodiments, the cancer is acute myeloid leukemia (AML).

Furthermore, in some aspects, the present invention includes a method for treating or preventing an autoimmune and/or neurodegenerative disease, which comprises administering an effective amount of the chimeric protein in accordance with various embodiments of the present disclosure to a patient in need thereof. The autoimmune and/or neurodegenerative disease can be selected from multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, myasthenia gravis, Reiter's syndrome, and Grave's disease.

In some embodiments, a chimeric protein is provided that comprises an amino acid sequence having at least 90% identity with SEQ ID NO: 9, or an amino acid sequence having at least 95% identity with SEQ ID NO: 9.

In some embodiments, the present chimeric protein optionally comprises one or more flexible linkers. In some embodiments, the present chimeric protein comprises a flexible linker connecting the targeting moiety and the signaling agent (e.g., IFNα2, IFNβ, or IL-1β or a variant thereof). In some embodiments, the present chimeric protein comprises a flexible linker within the signaling agent (e.g., IFNα2, IFNβ, or IL-1β or a variant thereof). In some embodiments, the flexible linker may be utilized to link various functional groups, residues, or moieties as described herein to the chimeric protein. In some embodiments, the flexible linker is a plurality of amino acids that does not affect or reduce the stability, orientation, binding, neutralization, and/or clearance characteristics of the binding regions and the binding protein.

In some embodiments, the chimeric protein comprises one or more additional signaling agents, e.g., without limitation, an interferon, an interleukin, as described herein, that may be wild type or modified. In various embodiments, the chimeric protein of the invention has a modified signaling agent and provides improved safety compared to an unmodified, wild type. For clarity, the present invention includes, in embodiments, chimeric proteins having one, or two, or three signaling agents.

In various embodiments, the chimeric protein comprises one or more targeting moieties which have targeting moiety (e.g., without limitation various antibody formats, inclusive of single-domain antibodies) which specifically bind to a target (e.g., antigen, receptor) of interest. In various embodiments, the targeting moieties specifically bind to a target (e.g., antigen, receptor) of interest, including those found on one or more immune cells, which can include, without limitation, T cells, cytotoxic T lymphocytes, T helper cells, natural killer (NK) cells, natural killer T (NKT) cells, anti-tumor macrophages (e.g., M1 macrophages), B cells, and dendritic cells. In some embodiments, the targeting moieties specifically bind to a target (e.g., antigen, receptor) of interest and effectively recruit one of more immune cells. In some embodiments, the targets (e.g., antigens, receptors) of interest can be found on one or more tumor cells. In some embodiments, the present chimeric proteins may recruit an immune cell, e.g., an immune cell that can kill and/or suppress a tumor cell, to a site of action (such as, by way of non-limiting example, the tumor microenvironment). In some embodiments, the targeting moieties specifically bind to a target (e.g., antigen, receptor) of interest which is part of a non-cellular structure. For clarity, the present invention includes, in embodiments, chimeric proteins having one, or two, or three targeting moieties.

In some embodiments vectors encoding the present chimeric proteins linked as a single nucleotide sequence to any of the flexible linkers described herein are provided and may be used to prepare such chimeric proteins. In some embodiments, the flexible linker length allows for efficient binding of a targeting moiety and the signaling agent (e.g., IFNα2, IFNβ, or IL-β or a variant thereof) to their receptors. For instance, in some embodiments, the flexible linker length allows for efficient binding of one of the targeting moieties and the signaling agent to receptors on the same cell.

In some embodiments the flexible linker length is at least equal to the minimum distance between the binding sites of one of the targeting moieties and the signaling agent to receptors on the same cell. In some embodiments the flexible linker length is at least twice, or three times, or four times, or five times, or ten times, or twenty times, or 25 times, or 50 times, or one hundred times, or more the minimum distance between the binding sites of one of the targeting moieties and the signaling agent to receptors on the same cell.

As described herein, the flexible linker length allows for efficient binding of one of the targeting moieties and the signaling agent to receptors on the same cell, the binding being sequential, e.g. targeting moiety/receptor binding preceding signaling agent/receptor binding.

In some embodiments, there are two flexible linkers in a single chimera, each connecting the signaling agent to a targeting moiety. In various embodiments, the flexible linkers have lengths that allow for the formation of a site that has a disease cell and an effector cell without steric hindrance that would prevent modulation of the either cell.

The invention contemplates the use of a variety of flexible linker sequences. In various embodiments, the flexible linker may be functional. For example, without limitation, the flexible linker may function to improve the folding and/or stability, improve the expression, improve the pharmacokinetics, and/or improve the bioactivity of the present chimeric protein.

In some embodiments, the linker is a polypeptide. In some embodiments, the flexible linker is less than about 100 amino acids long. For example, the flexible linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the flexible linker is a polypeptide. In some embodiments, the flexible linker is greater than about 100 amino acids long. For example, the flexible linker may be greater than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In various embodiments, the flexible linker is substantially comprised of glycine and serine residues (e.g. about 30%, or about 40%, or about 50%, or about 60%, or about 70%, or about 80%, or about 90%, or about 95%, or about 97% glycines and serines). For example, in some embodiments, the flexible linker is (Gly₄Ser)ₙ, where n is from about 1 to about 8, e.g. 1, 2, 3, 4, 5, 6, 7, or 8 (SEQ ID NO: 10-SEQ ID NO: 17, respectively). In an embodiment, the flexible linker sequence is GGSGGSGGGGSGGGGS (SEQ ID NO: 18). Additional illustrative flexible linkers include, but are not limited to, flexible linkers having the sequence LE, GGGGS (SEQ ID NO: 10), (GGGGS)ₙ(n=1-4) (SEQ ID NO: 10-SEQ ID NO: 13), (Gly)₈ (SEQ ID NO: 19), (Gly)₆ (SEQ ID NO: 20), (EAAAK)ₙ (n=1-3) (SEQ ID NO: 21-SEQ ID NO: 23), A(EAAAK)ₙA (n=2-5) (SEQ ID NO: 24-SEQ ID NO: 27), AEAAAKEAAAKA (SEQ ID NO: 24), A(EAAAK)₄ALEA(EAAAK)₄A (SEQ ID NO: 28), PAPAP (SEQ ID NO: 29), KESGSVSSEQLAQFRSLD (SEQ ID NO: 30), EGKSSGSGSESKST (SEQ ID NO: 31), GSAGSAAGSGEF (SEQ ID NO: 32), and (XP)ₙ, with X designating any amino acid, e.g., Ala, Lys, or Glu. In various embodiments, the flexible linker is GGS.

In some embodiments, the flexible linker is one or more of GGGSE (SEQ ID NO: 33), GSESG (SEQ ID NO: 34), GSEGS (SEQ ID NO: 35), GEGGSGEGSSGEGSSSEGGGSEGGGSEGGGSEGGS (SEQ ID NO: 36), and a flexible linker of randomly placed G, S, and E every 4 amino acid intervals.

In various embodiments, the flexible linker may be functional. For example, without limitation, the flexible linker may function to improve the folding and/or stability, improve the expression, improve the pharmacokinetics, and/or improve the bioactivity of the present chimeric protein. In another example, the flexible linker may function to target the chimeric protein to a particular cell type or location.

In various embodiments, the present chimeric protein may include one or more functional groups, residues, or moieties. In various embodiments, the one or more functional groups, residues, or moieties are attached or genetically fused to any of the signaling agents or targeting moieties described herein. In some embodiments, such functional groups, residues or moieties confer one or more desired properties or functionalities to the chimeric protein of the invention. Examples of such functional groups and of techniques for introducing them into the chimeric protein are known in the art, for example, see Remington's Pharmaceutical Sciences, 16th ed., Mack Publishing Co., Easton, Pa. (1980).

In various embodiments, each of the chimeric proteins may by conjugated and/or fused with another agent to extend half-life or otherwise improve pharmacodynamic and pharmacokinetic properties. In some embodiments, the chimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. p In various embodiments, each of the individual chimeric proteins is fused to one or more of the agents described in BioDrugs (2015) 29:215-239, the entire contents of which are hereby incorporated by reference.

In some embodiments, the functional groups, residues, or moieties comprise a suitable pharmacologically acceptable polymer, such as poly(ethyleneglycol) (PEG) or derivatives thereof (such as methoxypoly(ethyleneglycol) or mPEG). In some embodiments, attachment of the PEG moiety increases the half-life and/or reduces the immunogenecity of the chimeric protein. Generally, any suitable form of pegylation can be used, such as the pegylation used in the art for antibodies and antibody fragments (including but not limited to single domain antibodies such as VHHs); see, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO04060965, the entire contents of which are hereby incorporated by reference. Various reagents for pegylation of proteins are also commercially available, for example, from Nektar Therapeutics, USA. In some embodiments, site-directed pegylation is used, in particular via a cysteine-residue (see, for example, Yang et al., Protein Engineering, 16, 10, 761-770 (2003), the entire contents of which is hereby incorporated by reference). In some embodiments, the chimeric protein of the invention is modified so as to suitably introduce one or more cysteine residues for attachment of PEG, or an amino acid sequence comprising one or more cysteine residues for attachment of PEG may be fused to the amino-and/or carboxy-terminus of the chimeric proteins, using techniques known in the art.

In some embodiments, the functional groups, residues, or moieties comprise N-linked or 0-linked glycosylation. In some embodiments, the N-linked or 0-linked glycosylation is introduced as part of a co-translational and/or post-translational modification.

In some embodiments, the functional groups, residues, or moieties comprise one or more detectable labels or other signal-generating groups or moieties. Suitable labels and techniques for attaching, using and detecting them are known in the art and, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels include moieties that can be detected using NMR or ESR spectroscopy. Such labeled polypeptides of the invention may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label. In some embodiments, the functional groups, residues, or moieties comprise a tag that is attached or genetically fused to the chimeric protein. In some embodiments, the chimeric protein may include a single tag or multiple tags. The tag for example is a peptide, sugar, or DNA molecule that does not inhibit or prevent binding of the chimeric protein to its target or any other antigen of interest such as tumor antigens. In various embodiments, the tag is at least about: three to five amino acids long, five to eight amino acids long, eight to twelve amino acids long, twelve to fifteen amino acids long, or fifteen to twenty amino acids long. Illustrative tags are described for example, in U.S. Patent Publication No. US2013/0058962. In some embodiment, the tag is an affinity tag such as glutathione-S-transferase (GST) and histidine (His) tag. In an embodiment, the chimeric protein comprises a His tag.

In some embodiments, the functional groups, residues, or moieties comprise a chelating group, for example, to chelate one of the metals or metallic cations. Suitable chelating groups, for example, include, without limitation, diethyl-enetriaminepentaacetic acid (DTPA) or ethylenediaminetetraacetic acid (EDTA).

In some embodiments, the functional groups, residues, or moieties comprise a functional group that is one part of a specific binding pair, such as the biotin-(strept)avidin binding pair. Such a functional group may be used to link the chimeric protein of the invention to another protein, polypeptide or chemical compound that is bound to the other half of the binding pair, i.e., through formation of the binding pair. For example, a chimeric protein of the invention may be conjugated to biotin, and linked to another protein, polypeptide, compound or carrier conjugated to avidin or streptavidin. For example, such a conjugated chimeric protein may be used as a reporter, for example, in a diagnostic system where a detectable signal-producing agent is conjugated to avidin or streptavidin. Such binding pairs may, for example, also be used to bind the chimeric protein to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the chimeric protein of the invention.

Methods for producing the chimeric proteins of the invention are described herein. For example, DNA sequences encoding the chimeric proteins of the invention (e.g., DNA sequences encoding the signaling agent (e.g., IFNα2, IFNβ, or IL-1β or a variant thereof) and the targeting moiety and the flexible linker) can be chemically synthesized using methods known in the art. Synthetic DNA sequences can be ligated to other appropriate nucleotide sequences, including, e.g., expression control sequences, to produce gene expression constructs encoding the desired chimeric proteins. Accordingly, in various embodiments, the present invention provides for isolated nucleic acids comprising a nucleotide sequence encoding the chimeric protein of the invention.

Nucleic acids encoding the chimeric protein of the invention can be incorporated (ligated) into expression vectors, which can be introduced into host cells through transfection, transformation, or transduction techniques. For example, nucleic acids encoding the chimeric protein of the invention can be introduced into host cells by retroviral transduction. Illustrative host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the chimeric protein of the invention. Accordingly, in various embodiments, the present invention provides expression vectors comprising nucleic acids that encode the chimeric protein of the invention. In various embodiments, the present invention additional provides host cells comprising such expression vectors.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. In another example, if the engineered gene is to be expressed in eukaryotic host cells, e.g., CHO cells, it is first inserted into an expression vector containing for example, a suitable eukaryotic promoter, a secretion signal, enhancers, and various introns. The gene construct can be introduced into the host cells using transfection, transformation, or transduction techniques.

The chimeric protein of the invention can be produced by growing a host cell transfected with an expression vector encoding the chimeric protein under conditions that permit expression of the protein. Following expression, the protein can be harvested and purified using techniques well known in the art, e.g., affinity tags such as glutathione-S-transferase (GST) and histidine tags or by chromatography.

Accordingly, in various embodiments, the present invention provides for a nucleic acid encoding a chimeric protein of the present invention. In various embodiments, the present invention provides for a host cell comprising a nucleic acid encoding a chimeric protein of the present invention. In various embodiments, the present invention provides nucleic acid encoding a chimeric protein of the present invention which is suitable for production in a non-cellular system (e.g. in vitro transcription and/or in vitro translation).

In various embodiments, IFNα2, IFNβ, or IL-1β, its variant, or a chimeric protein comprising the IFNα2, IFNβ, or IL-1β or its variant may be expressed in vivo, for instance, in a patient. For example, in various embodiments, the IFNα2, IFNβ, or IL-1β, its variant, or a chimeric protein comprising the IFNα2, IFNβ, or IL-1β or its variant may administered in the form of nucleic acid which encodes for the IFNα2, IFNβ, or IL-1β or its variant or chimeric proteins comprising IFNα2, IFNβ, or IL-1β or its variant. In various embodiments, the nucleic acid is DNA or RNA. In some embodiments, the IFNα2, IFNβ, or IL-1β, its variant, or a chimeric protein comprising the IFNα2, IFNβ, or IL-1β or its variant is encoded by a modified mRNA, i.e. an mRNA comprising one or more modified nucleotides. In some embodiments, the modified mRNA comprises one or modifications found in U.S. Pat. No. 8,278,036, the entire contents of which are hereby incorporated by reference. In some embodiments, the modified mRNA comprises one or more of m5C, m5U, m6A, s2U, Ψ, and 2'-O-methyl-U. In some embodiments, the present invention relates to administering a modified mRNA encoding one or more of the present chimeric proteins. In some embodiments, the present invention relates to gene therapy vectors comprising the same. In some embodiments, the present invention relates to gene therapy methods comprising the same. In various embodiments, the nucleic acid is in the form of an oncolytic virus, e.g. an adenovirus, reovirus, measles, herpes simplex, Newcastle disease virus or vaccinia.

The chimeric proteins described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartrate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-, bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

In various embodiments, the present invention pertains to pharmaceutical compositions comprising the chimeric proteins described herein and a pharmaceutically acceptable carrier or excipient. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid. Where necessary, the inventive pharmaceutical compositions (and/or additional agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In some embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In one embodiment, the chimeric protein described herein is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any chimeric proteins described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof. The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like. Any inventive pharmaceutical compositions (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled-or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

It will be appreciated that the actual dose of the chimeric protein to be administered according to the present invention will vary according to the particular dosage form, and the mode of administration. Many factors that may modify the action of the chimeric protein (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In some embodiments, a suitable dosage of the chimeric protein is in a range of about 0.01 μg/kg to about 100 mg/kg of body weight of the subject, about 0.01 μg/kg to about 10 mg/kg of body weight of the subject, or about 0.01 μg/kg to about 1 mg/kg of body weight of the subject for example, about 0.01 μg/kg, about 0.02 μg/kg, about 0.03 μg/kg, about 0.04 μg/kg, about 0.05 μg/kg, about 0.06 μg/kg, about 0.07 μg/kg, about 0.08 μg/kg, about 0.09 μg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, or about 100 mg/kg body weight, inclusive of all values and ranges therebetween.

Individual doses of the chimeric protein can be administered in unit dosage forms (e.g., tablets, capsules, or liquid formulations) containing, for example, from about 1 μg to about 100 mg, from about 1 μg to about 90 mg, from about 1 μg to about 80 mg, from about 1 μg to about 70 mg, from about 1 μg to about 60 mg, from about 1 μg to about 50 mg, from about 1 μg to about 40 mg, from about 1 μg to about 30 mg, from about 1 μg to about 20 mg, from about 1 μg to about 10 mg, from about 1 μg to about 5 mg, from about 1 μg to about 3 mg, from about 1 μg to about 1 mg per unit dosage form, or from about 1 μg to about 50 μg per unit dosage form. For example, a unit dosage form can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In one embodiment, the chimeric protein is administered at an amount of from about 1 μg to about 100 mg daily, from about 1 μg to about 90 mg daily, from about 1 μg to about 80 mg daily, from about 1 μg to about 70 mg daily, from about 1 μg to about 60 mg daily, from about 1 μg to about 50 mg daily, from about 1 μg to about 40 mg daily, from about 1 μg to about 30 mg daily, from about 1 μg to about 20 mg daily, from about 01 μg to about 10 mg daily, from about 1 μg to about 5 mg daily, from about 1 μg to about 3 mg daily, or from about 1 μg to about 1 mg daily. In various embodiments, the chimeric protein is administered at a daily dose of about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg about 21 μg, about 22 μg, about 23 μg, about 24 μg, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In accordance with certain embodiments of the invention, the pharmaceutical composition comprising the chimeric protein may be administered, for example, more than once daily (e.g., about two times, about three times, about four times, about five times, about six times, about seven times, about eight times, about nine times, or about ten times daily), about once per day, about every other day, about every third day, about once a week, about once every two weeks, about once every month, about once every two months, about once every three months, about once every six months, or about once every year. In an embodiment, the pharmaceutical composition comprising the chimeric protein is administered about three times a week.

In various embodiments, the present chimeric protein may be administered for a prolonged period. For example, the chimeric protein may be administered as described herein for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks. For example, the chimeric protein may be administered for 12 weeks, 24 weeks, 36 weeks or 48 weeks. In some embodiments, the chimeric protein is administered for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. n some embodiments, the chimeric protein may be administered for at least about 1 year, at least about 2 years, at least about 3 years, at least about 4 years, or at least about 5 years.

In various embodiments, the pharmaceutical composition of the present invention is co-administered in conjunction with additional therapeutic agent(s). Co-administration can be simultaneous or sequential.

In one embodiment, the additional therapeutic agent and the chimeric protein of the present invention are administered to a subject simultaneously. The term “simultaneously” as used herein, means that the additional therapeutic agent and the chimeric protein are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the chimeric protein can be by simultaneous administration of a single formulation (e.g., a formulation comprising the additional therapeutic agent and the chimeric protein) or of separate formulations (e.g., a first formulation including the additional therapeutic agent and a second formulation including the chimeric protein).

Co-administration does not require the therapeutic agents to be administered simultaneously, if the timing of their administration is such that the pharmacological activities of the additional therapeutic agent and the chimeric protein overlap in time, thereby exerting a combined therapeutic effect. For example, the additional therapeutic agent and the chimeric protein can be administered sequentially. The term “sequentially” as used herein means that the additional therapeutic agent and the chimeric protein are administered with a time separation of more than about 60 minutes. For example, the time between the sequential administration of the additional therapeutic agent and the chimeric protein can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the chimeric protein being administered. Either the additional therapeutic agent or the chimeric protein cell may be administered first.

Co-administration also does not require the therapeutic agents to be administered to the subject by the same route of administration. Rather, each therapeutic agent can be administered by any appropriate route, for example, parenterally or non-parenterally.

In some embodiments, the chimeric protein described herein acts synergistically when co-administered with another therapeutic agent. In such embodiments, the chimeric protein and the additional therapeutic agent may be administered at doses that are lower than the doses employed when the agents are used in the context of monotherapy.

In some embodiments, the present invention pertains to chemotherapeutic agents as additional therapeutic agents. For example, without limitation, such combination of the present chimeric proteins and chemotherapeutic agent find use in the treatment of cancers, as described elsewhere herein. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino- doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In some embodiments, the chimeric protein described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc. In still other embodiments, the chimeric protein described herein further comprise a cytotoxic agent, comprising, in illustrative embodiments, a toxin, a chemotherapeutic agent, a radioisotope, and an agent that causes apoptosis or cell death. Such agents may be conjugated to a composition described herein.

The chimeric protein described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Illustrative cytotoxic agents include, but are not limited to, methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine. Other cytotoxic agents include paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O, P′-(DDD)), interferons, and mixtures of these cytotoxic agents. Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1 antagonists, interleukins (e.g. IL-12 or IL-2), IL-12R antagonists, Toxin conjugated monoclonal antibodies, tumor antigen specific monoclonal antibodies, Erbitux, Avastin, Pertuzumab, anti-CD20 antibodies, Rituxan, ocrelizumab, ofatumumab, DXL625, HERCEPTIN®, or any combination thereof. Toxic enzymes from plants and bacteria such as ricin, diphtheria toxin and Pseudomonas toxin may be conjugated to the therapeutic agents (e.g. antibodies) to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)).

Other cytotoxic agents include cytotoxic ribonucleases as described by Goldenberg in U.S. Pat. No. 6,653,104. Embodiments of the invention also relate to radioimmunoconjugates where a radionuclide that emits alpha or beta particles is stably coupled to the chimeric protein, with or without the use of a complex-forming agent. Such radionuclides include beta-emitters such as Phosphorus-32, Scandium-47, Copper-67, Gallium-67, Yttrium-88, Yttrium-90, Iodine-125, Iodine-131, Samarium-153, Lutetium-177, Rhenium-186 or Rhenium-188, and alpha-emitters such as Astatine-211, Lead-212, Bismuth-212, Bismuth-213 or Actinium-225.

Illustrative detectable moieties further include, but are not limited to, horseradish peroxidase, acetylcholinesterase, alkaline phosphatase, beta-galactosidase and luciferase. Further illustrative fluorescent materials include, but are not limited to, rhodamine, fluorescein, fluorescein isothiocyanate, umbelliferone, dichlorotriazinylamine, phycoerythrin and dansyl chloride. Further illustrative chemiluminescent moieties include, but are not limited to, luminol. Further illustrative bioluminescent materials include, but are not limited to, luciferin and aequorin. Further illustrative radioactive materials include, but are not limited to, Iodine-125, Carbon-14, Sulfur-35, Tritium and Phosphorus-32.

In some embodiments, inclusive, without limitation, of autoimmune applications, the additional therapeutic agent is an immunosuppressive agent that is an anti-inflammatory agent such as a steroidal anti-inflammatory agent or a non-steroidal anti-inflammatory agent (NSAID). Steroids, particularly the adrenal corticosteroids and their synthetic analogues, are well known in the art. Examples of corticosteroids useful in the present invention include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. In some embodiments, the immunosupressive agent may be cytostatics such as alkylating agents, antimetabolites (e.g., azathioprine, methotrexate), cytotoxic antibiotics, antibodies (e.g., basiliximab, daclizumab, and muromonab), anti-immunophilins (e.g., cyclosporine, tacrolimus, sirolimus), inteferons, opioids, TNF binding proteins, mycophenolates, and small biological agents (e.g., fingolimod, myriocin). Additional anti-inflammatory agents are described, for example, in U.S. Pat. No. 4,537,776, the entire contents of which is incorporated by reference herein.

In some embodiments, the chimeric protein is used in a method of treating multiple sclerosis in combination with one or more of the disease modifying therapies (DMTs) described herein (e.g. the agents of Table A). In some embodiments, the present invention provides an improved therapeutic effect as compared to use of one or more of the DMTs described herein (e.g. the agents listed in Table A below) without the one or more disclosed binding agent. In an embodiment, the combination of the chimeric protein and the one or more DMTs produces synergistic therapeutic effects.

Illustrative disease modifying therapies include, but are not limited to:

TABLE A

| Generic Name | Branded Name/Company | Frequency/Route of Delivery/Usual Dose |
|---|---|---|
| teriflunomide | AUBAGIO (GENZYME) | Every day; pill taken orally; 7 mg or 14 mg. |
| interferon beta-1a | AVONEX (BIOGEN IDEC) | Once a week; intramuscular (into the muscle) |
| | | injection; 30 mcg |
| interferon beta-1b | BETASERON (BAYER | Every other day; subcutaneous (under the skin) |
| | HEALTHCARE | injection; 250 mcg. |
| | PHARMACEUTICALS, | |
| | INC.) | |
| glatiramer acetate | COPAXONE (TEVA | Every day; subcutaneous (under the skin) injection; |
| | NEUROSCIENCE) | 20 mg (20,000 mcg) OR Three times a week; |
| | | subcutaneous (under the skin) injection; 40 mg |
| | | (40,000 mcg) |
| interferon beta-1b | EXTAVIA (NOVARTIS | Every other day; subcutaneous (under the skin) |
| | PHARMACEUTICALS | injection; 250 mcg. |
| | CORP.) | |
| fingolimod | GILENYA (NOVARTIS | Every day; capsule taken orally; 0.5 mg. |
| | PHARMACEUTICALS | |
| | CORP.) | |
| Alemtuzumab (anti-CD52 | LEMTRADA (GENZYME) | Intravenous infusion on five consecutive days, |
| monoclonal antibody) | | followed by intravenous infusion on three |
| | | consecutive days one year later (12 mg) |
| mitoxantrone | NOVANTRONE (EMD | Four times a year by IV infusion in a medical facility. |
| | SERONO) | Lifetime cumulative dose limit of approximately 8- |
| | | 12 doses over 2-3 years (140 mg/m2). |
| pegylated interferon beta-1a | PLEGRIDY (BIOGEN | Every 14 days; subcutaneous (under the skin) |
| | IDEC) | injection; 125 mcg |
| interferon beta-1a | REBIF (EMD SERONO, | Three times a week; subcutaneous (under the skin) |
| | INC.) | injection; 44 mcg |
| dimethyl fumarate (BG-12) | TECFIDERA (BIOGEN | Twice a day; capsule taken orally; 120 mg for one |
| | IDEC) | week and 240 mg therafter |
| Natalizumab (humanized | TYSABRI (BIOGEN IDEC) | Every four weeks by IV infusion in a registered |
| monoclonal antibody VLA-4 | | infusion facility; 300 mg |
| antagonist) | | |

DMTs in Development

| | | |
|---|---|---|
| Amiloride (targets Acid-sensing | PAR PHARMACEUTICAL, | Oral |
| ion channel-1 Epithelial sodium | PERRIGO COMPANY, | |
| channel Na+/H+exchanger) | SIGMAPHARM | |
| | LABORATORIES | |
| ATX-MS-1467 (targets Major | APITOPE/MERCK | Intradermal Subcutaneous |
| histocompatibility complex class II | SERONO | |
| T cell responses to myelin basic | | |
| protein) | | |
| BAF312 (targets Sphingosine 1- | NOVARTIS PHARMA | Oral |
| phosphate (S1P) receptor | | |
| subtypes S1P1 and S1P5 B cell | | |
| distrubution T cell distribution) | | |
| BGC20-0134 (targets | BTG PLC | Oral |
| Proinflammatory and anti- | | |
| inflammatory cytokines) | | |
| BIIB033 (targets LINGO-1 | BIOGEN | Intravenous infusion used in Phase I and Phase II |
| (“leucine-rich repeat and | | trials Subcutaneous injection used in Phase I trial |
| immunoglobulin-like domain- | | |
| containing, Nogo receptor- | | |
| interacting protein”)) | | |
| Cladribine (targets CD4+ T cells | MERCK SERONO | Oral |
| DNA synthesis and repair E- | | |
| selectin Intracellular adhesion | | |
| molecule-1 Pro-inflammatory | | |
| cytokines interleukin 2 and | | |
| interleukin 2R Pro-inflammatory | | |
| cytokines interleukin 8 and | | |
| RANTES Cytokine secretion | | |
| Monocyte and lymphocyte | | |
| migration) | | |
| Cyclophosphamide (targets T | BAXTER HEALTHCARE | Oral, monthly intravenous pulses |
| cells, particularly CD4+ helper T | CORPORATION | |
| cells B cells) | | |
| Daclizumab (humanized | BIOGEN IDEC/ABBVIE | Projected to be IM injection once monthly |
| monoclonal antibody targeting | BIOTHERAPEUTICS | |
| CD25 Immune modulator of T | | |
| cells) | | |
| Dalfampridine (targets Voltage- | ACORDA | One tablet every 12 hours (extended release), 10 |
| gated potassium channels | THERAPEUTICS/ | mg twice a day |
| Degenerin/epithelial sodium | BIOGEN IDEC | |
| channels L-type calcium channels | | |
| that contain subunit Cavbeta3) | | |
| Dronabinol (targets Cannabinoid | ABBVIE INC. | Oral |
| receptor CB1 Cannabinoid | | |
| receptor CB2) | | |
| Firategrast (targets Alpha4beta1 | GLAXOSMITHKLINE | Oral |
| integrin) | | |
| GNbAC1MSRV-Env (targets | GENEURO SA/SERVIER | Intravenous infusion |
| envelope protein of the MS- | | |
| associated retrovirus) | | |
| Idebenone (targets Reactive | SANTHERA | Oral Dose in clinical trial for PPMS is 2250 mg per |
| oxygen species) | PHARMACEUTICALS | day (750 mg dose, 3 times per day) |
| Imilecleucel-T (targets Myelin- | OPEXA THERAPEUTICS/ | Subcutaneous Given 5 times per year, according to |
| specific, autoreactive T cells) | MERCK SERONO | information from the manufacturer |
| Laquinimod | TEVA | Projected to be 0.6 mg or 1.2 mg oral tablet taken |
| | | daily |
| Masitinib (targets KIT (a stem cell | AB SCIENCE | Oral |
| factor, also called c-KIT) receptor | | |
| as well as select other tyrosine | | |
| kinases Mast cells) | | |
| MEDI-551 (targets CD19, a B cell- | MEDIMMUNE | Intravenous Subcutaneous |
| specific antigen that is part of the B | | |
| cell receptor complex and that | | |
| functions in determining the | | |
| threshold for B cell activation B | | |
| cells Plasmablasts, B cells that | | |
| express CD19 (but not CD20) and | | |
| that secrete large quantities of | | |
| antibodies; depletion of | | |
| plasmablasts may be useful in | | |
| autoimmune diseases involving | | |
| pathogenic autoantibodies) | | |
| Minocycline (targets T cells | VARIOUS | Oral Available as pellet-filled capsules and an oral |
| Microglia Leukocyte migration | | suspension |
| Matrix metalloproteinases) | | |
| MIS416 (targets Innate immune | INNATE | Intravenous |
| system Pathogen-associated | IMMUNOTHERAPEUTICS | |
| molecular pattern recognition | | |
| receptors of the innate immune | | |
| system Myeloid cells of the innate | | |
| immune system, which might be | | |
| able to remodel the deregulated | | |
| immune system activity that occurs | | |
| in SPMS) | | |
| Mycophenolate mofetil (targets | MANUFACTURED BY | Oral |
| Purine synthesis) | GENENTECH | |
| Naltrexone (targets Opioid | VARIOUS | Given at low doses (3 to 4.5 mg per day) in oral |
| receptors Toll-like receptor 4) | | form as “Low-dose naltrexone” (or “LDN”) |
| Ocrelizumab and Ofatumumab | ROCHE/GSK | Projected to be IV infusion |
| (humanized monoclonal | | |
| antibodies targeting CD20 B cell | | |
| suppression | | |
| ONO-4641 (targets Sphingosine | ONO PHARMACEUTICAL | Oral |
| 1-phosphate receptor) | CO. | |
| Phenytoin (targets Sodium | PFIZER | Intravenous Intramuscular (less favored option) |
| channels) | | Oral |
| Ponesimod | ACTELION | To be determined |
| Raltegravir (targets Retroviral | MERCK | Oral 400 mg tablet twice daily, according to |
| integrase Herpesvirus DNA | | information from the manufacturer |
| packaging terminase) | | |
| RHB-104 | REDHILL BIOPHARMA | 95 mg clarithromycin, 45 mg rifabutin, and 10 mg |
| | LIMITED | clofazimine |
| Riluzole (targets Glutamatergic | COVIS PHARMA/ | Oral |
| neurotransmission Glutamate | SANOFI | |
| uptake and release Voltage-gated | | |
| sodium channels Protein kinase C) | | |

The invention also provides kits for the administration of any agent described herein (e.g. the chimeric protein with or without various additional therapeutic agents). The kit is an assemblage of materials or components, including at least one of the inventive pharmaceutical compositions described herein. Thus, in some embodiments, the kit contains at least one of the pharmaceutical compositions described herein.

The exact nature of the components configured in the kit depends on its intended purpose. In one embodiment, the kit is configured for the purpose of treating human subjects.

Instructions for use may be included in the kit. Instructions for use typically include a tangible expression describing the technique to be employed in using the components of the kit to effect a desired outcome, such as to treat cancer. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner stored in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

Definitions

As used herein, “a,” “an,” or “the” can mean one or more than one.

Unless specifically stated or obvious from context, as used herein, the term “or” is understood to be inclusive and covers both “or” and “and”.

Further, the term “about” when used in connection with a referenced numeric indication means the referenced numeric indication plus or minus up to 10% of that referenced numeric indication, e.g., within (plus or minus) 10%, 9%, 8%, 7%, 6%, 5%, 4%, 3%, 2%, 1%, 0.5%, 1%, 0.05%, or 0.01% of the stated value. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the 1050 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein. This invention is further illustrated by the following non-limiting examples.

### EXAMPLES

“AFN” or “AcTaferon” is used occasionally herein to refer to an interferon-based chimeric protein described herein.

### Example 1: Flt3-Targeted AcTaferon In Vivo Study

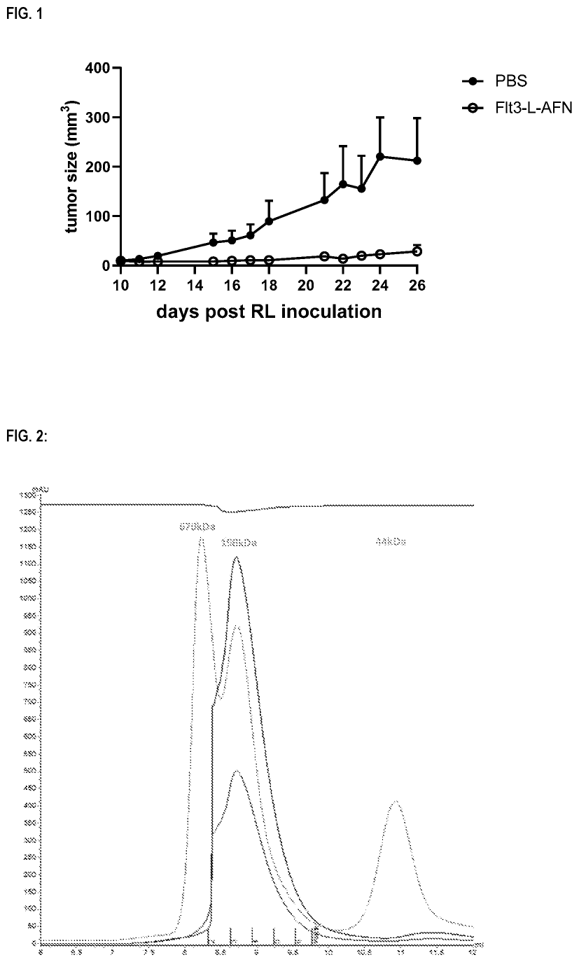

To evaluate the in vivo efficacy of Flt3 targeted AcTaferons, a Flt3L_ linker_humanIFNα2(R149A)_GGS_his9 fusion protein was expressed in HEKT cells and purified by metal affinity chromatography. The purified protein was subsequently evaluated in a tumor model in a humanized mouse. In brief, newborn NSG mice (1-2 days of age) were sublethal irradiated with 100 cGy prior to intrahepatic delivery of 10⁵ CD34+human stem cells (from HLA-A2 positive cord bloods). At week 13 after stem cell transfer mice were subcutaneously inoculated with 25.10E5 human RL follicular lymphoma cells (ATCC CRL-2261; not sensitive to the direct anti-proliferative effect of IFN). Mice were treated daily intraperitoneally with 30 μg of human Flt3L protein, from day 8 to day 18 after tumor inoculation. Daily perilesional injection with buffer or Flt3L-AFN (30 μg) was initiated at day 10 after tumor inoculation, when a palpable tumor was visible (n=5 or 6 mice per group). Tumor size (caliper measurements), body weight and temperature were assessed daily. Data in FIG. 1 show the tumor growth until 2 days after the last treatment and demonstrates that a Flt3 targeted AcTaferon strongly suppresses tumor growth. Data on body weight and temperature did not show any major difference between buffer treatment and AFN treatment supporting that the AFN treatment was well tolerated.

The Sequence mature Flt3L (bold)_linker (italics)_humanIFNa2(R149A) (italics and bold)GGS_his9 used in this example is:

(SEQ ID NO: 8)
TQDCSFQHSPISSDFAVKIRELSDYLLQDYPVTVASNLQDEELCGGLWRL
VLAQRWMERLKTVAGSKMQGLLERVNTEIHFVTKCAFQPPPSCLRFVQTN
ISRLLQETSEQLVALKPWITRQNFSRCLELQCQPDSSTLPPPWSPRPLEA
TAPTAVDGGSGGSGGSGGSGGSGGSRSGGSGGSGGSGGSGGSGGSGGSGG
SGGSGGSGGSGGSGGSAAAM

[symbol]

[symbol]

[symbol]

[symbol]

[symbol]

[symbol]

[symbol]

GGSHHHHHHHHH.

Without the His9, this sequence is:

(SEQ ID NO: 9)
TQDCSFQHSPISSDFAVKIRELSDYLLQDYPVTVASNLQDEELCGGLWRL
VLAQRWMERLKTVAGSKMQGLLERVNTEIHFVTKCAFQPPPSCLRFVQTN
ISRLLQETSEQLVALKPWITRQNFSRCLELQCQPDSSTLPPPWSPRPLEA
TAPTAVDGGSGGSGGSGGSGGSGGSRSGGSGGSGGSGGSGGSGGSGGSGG
SGGSGGSGGSGGSGGSAAAM

[symbol]

[symbol]

[symbol]

[symbol]

[symbol]

[symbol]

[symbol]

GGS.

### Example 2: FLT3L-AFN Fusion Forms a Dimer

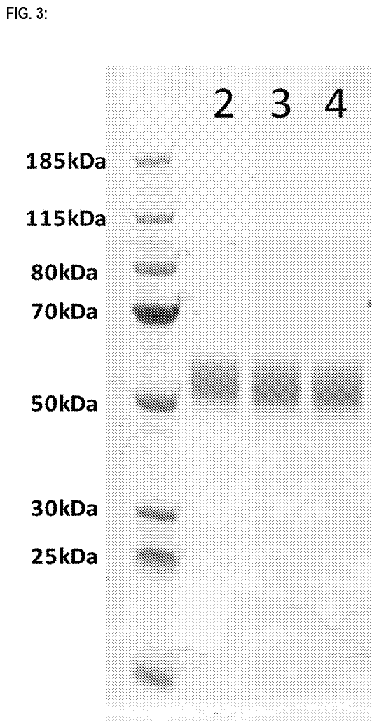

The Flt3L_ linker_human IFNα2(R149A)_GGS_his9 fusion protein of Example 1 was recloned into a pcDNA 3.4 vector for expression in CHO cells resulting in plasmid P-2373. Production was performed in ExpiCHO cells (ThermoFisher) according to the manufacturer's instructions. Seven days post transfection, supernatant was harvested and cells removed by centrifugation. Proteins were purified on a 1 ml HisTrap Excel column (GE Healthcare) on an AKTA pure instrument (GE Healthcare). Eluted protein was desalted by Sephadex G25 (5 ml column) to a PBS -H 8.0 buffer. Finally, the sample was further analyzed using size exclusion chromatography on the AKTA on a Superdex 75 Increase 10/300 column (GE Healthcare) in a 10 mM NH₄-acetate pH 5.0 buffer with 123.5 mM NaCl. The SEC profile and the subsequent analysis by SDS-PAGE of the peak fraction illustrate that the protein behaves in SEC (FIG. 2) as an about 150 kD protein (i.e. eluting at the 158 kD marker) while it behaves as an about 55 kD protein in SDS-PAGE (FIG. 3). These data support the formation of non-covalently linked dimer. Such dimers have not been seen with VHH based AFNs and thus are the consequence of an FLT3L dimerization as required for FLT3L receptor binding and signaling.

What is claimed is:

1. A chimeric protein comprising:
(i) a targeting moiety which comprises a single copy of FMS-like tyrosine kinase 3 ligand (FLT3L), or a portion thereof;
(ii) one or more flexible linkers connecting elements (i) and (iii); and
(iii) a signaling agent or a modified form thereof.

2. The chimeric protein of claim 1, wherein the targeting moiety comprises an amino acid sequence which is a truncation of SEQ ID NO: 1.

3. The chimeric protein of claim 1 or 2, wherein the targeting moiety comprises the extracellular domain of FLT3L, or a portion thereof.

4. The chimeric protein of any one of claims 1-3, wherein the targeting moiety comprises the extracellular domain of FLT3L.

5. The chimeric protein of claim 4, wherein the targeting moiety comprises an amino acid sequence having at least 90% identity with any one of SEQ ID NOs: 2-5.

6. The chimeric protein of claim 5, wherein the targeting moiety comprises an amino acid sequence having at least 95% identity with any one of SEQ ID NOs: 2-5.

7. The chimeric protein of claim 5, wherein the targeting moiety comprises an amino acid sequence having at least 97% identity with any one of SEQ ID NOs: 2-5.

8. The chimeric protein of any one of claims 1-7, wherein the signaling agent is wild type human IFNα2, IFNβ, or IL-1β.

9. The chimeric protein of any one of claims 1-8, wherein the signaling agent comprises an amino acid sequence having at least 95% identity with any one of SEQ ID NO: 6, 7, 38, or 39.

10. The chimeric protein of any one of claims 1-9, wherein the signaling agent comprises an amino acid sequence of any one of SEQ ID NO: 6, 7, 38, or 39.

11. The chimeric protein of any one of claims 1-10, wherein the signaling agent is modified to comprise one or more mutations.

12. The chimeric protein of claim 11, wherein the one or more mutations confer improved safety as compared to a wild type signaling agent.

13. The chimeric protein of claim 11, wherein the one or more mutations confer reduced affinity for the signaling agent's receptor.

14. The chimeric protein of claim 11, wherein the one or more mutations confer reduced bioactivity for the signaling agent's receptor.

15. The chimeric protein of any one of claims 11-14, wherein the one or more mutations allow for attenuation of the signaling agent's activity.

16. The chimeric protein of claim 15, wherein agonistic or antagonistic activity of the signaling agent is attenuated.

17. The chimeric protein of any one of claims 11-16, wherein the modified signaling agent comprises one or more mutations which convert its activity from agonistic to antagonistic.

18. The chimeric protein of any one of claims 11-17, wherein the one or more mutations confer reduced affinity or activity that is restorable by attachment to one or more targeting moiety.

19. The chimeric protein of any one of claims 11-18, wherein the one or more mutations confer substantially reduced or ablated affinity or activity that is not substantially restorable by attachment to a targeting moiety.

20. The chimeric protein of any one of claims 1-19, wherein the signaling agent is a mutant human IFNα2 comprising an amino acid sequence having at least 95% identity with SEQ ID NO: 6 or 7 and wherein the mutant human IFNα2 has one or more mutations that confer improved safety as compared to a wild type IFNα2 having an amino acid sequence of SEQ ID NO: 6 or 7.

21. The chimeric protein of claim 20, wherein the human IFNα2 has one or more mutations at positions 144 to 154 with respect to SEQ ID NO: 6 or 7.

22. The chimeric protein of claim 21, wherein the human IFNα2 has: one or more mutations at positions L15, A19, R22, R23, L26, F27, L30, L30, K31, D32, R33, H34, D35, Q40, H57, E58, Q61, F64, N65, T69, L80, Y85, Y89, D114, L117, R120, R125, K133, K134, R144, A145, M148, R149, S152, L153, and N156 with respect to SEQ ID NO: 6 or 7.

23. The chimeric protein of claim 21, wherein the mutant human IFNα2 has one or more mutations at position R33, T106, R144, A145, M148, R149, and L153 with respect to SEQ ID NO: 6 or 7.

24. The chimeric protein of claim 22, wherein the mutation is one or more of L15A, A19W, R22A, R23A, L26A, F27A, L30A, L30V, K31A, D32A, R33K, R33A, R33Q, H34A, D35A, Q40A, H57Y, E58N, Q61S, F64A, N65A, T69A, L80A, Y85A, Y89A, D114R, L117A, R120A, R125A, K133A, K134A, R144A, A145G, A145M, M148A, R149A, S152A, L153A, and N156A with respect to SEQ ID NO: 6 or 7.

25. The chimeric protein of claim 23, wherein the mutant human IFNα2 has one or more mutations selected from R33A, T106X₃, R120E, R144X₁ A145X₂, M148A, R149A, and L153A with respect to amino acid sequence of SEQ ID NO: 6 or 7, wherein X₁ is selected from A, S, T, Y, L, and I, wherein X2 is selected from G, H, Y, K, and D, and wherein X3 is selected from A and E.

26. The chimeric protein of any one of claims 1-19, wherein the signaling agent is a mutant human IFNβ comprising an amino acid sequence having at least 95% identity with SEQ ID NO: 38 and wherein the mutant human IFNβ has one or more mutations that confer improved safety as compared to a wild type IFNβ having an amino acid sequence of SEQ ID NO: 38.

27. The chimeric protein of claim 26, wherein the mutation is one or more of W22G, R27G, L32A, L32G, R35A, R35G, V148G, L151G, R152A, R152G with respect to amino acid sequence of SEQ ID NO: 38.

28. The chimeric protein of any one of claims 1-19, wherein the signaling agent is a mutant human IL-1β comprising an amino acid sequence having at least 95% identity with SEQ ID NO: 39 and wherein the mutant human IL-1β has one or more mutations that confer improved safety as compared to a wild type IL-1β having an amino acid sequence of SEQ ID NO: 39.

29. The chimeric protein of claim 28, wherein the mutation is one or more of A117G/P118G, R120G, R120A, L122A, T125G/L126G, R127G, Q130A, Q130W, Q131G, K132A, S137G/Q138Y, L145G, H146A, H146G, H146E, H146N, H146R, L145A/L147A, Q148E, Q148G, Q148L, Q148G/Q150G, Q150G/D151A, M152G, F162A, F162A/Q164E, F166A, Q164E/E167K, N169G/D170G, 1172A, V174A, K208E, K209A, K209D, K209A/K210A, K219S, K219Q, E221S, E221K, E221S/N224A, N224S/K225S, E244K and N245Q with respect to amino acid sequence of SEQ ID NO: 39.

30. The chimeric protein of any one of the above claims, wherein the targeting moiety is directed against an immune cell, optionally being a dendritic cell.

31. The chimeric protein of claim 30, wherein the dendritic cell is a conventional dendritic cell (cDC), optionally being a cDC-1, migratory DC, and Flt3+ DC.

32. The chimeric protein of claim 31, wherein the targeting moiety is directed to a hematopoietic stem cell (HSC), early progenitor cell, immature thymocyte, or steady state dendritic cell (DC).

33. The chimeric protein of any one of the above claims, wherein the targeting moiety functionally modulates the antigen or receptor of interest.

34. The chimeric protein of any one of the above claims, wherein the targeting moiety binds but does not functionally modulate the antigen or receptor of interest.

35. The chimeric protein of any one of the above claims, wherein the targeting moiety directly or indirectly recruits immune cells to tumor cells or to the tumor microenvironment.

36. The chimeric protein of any one of the above claims, wherein the targeting moiety increases a number of dendritic cells.

37. The chimeric protein of any one of the above claims, wherein the targeting moiety enhances tumor antigen presentation, optionally by dendritic cells.

38. The chimeric protein of any one of the above claims, comprising two targeting moieties, which are identical or non-identical.

39. The chimeric protein of any one of the above claims, comprising an additional signaling agent.

40. The chimeric protein of any one of the above claims, comprising two signaling agents.

41. The chimeric protein of any one of the above claims, wherein the flexible linker is substantially comprised of glycine and serine residues.

42. The chimeric protein of any one of the above claims, wherein the flexible linker comprises (Gly₄Ser)ₙ, where n is from about 1 to about 8.

43. The chimeric protein of any one of the above claims, wherein the flexible linker comprises one or more of SEQ ID NO: 10-SEQ ID NO: 17.

44. The chimeric protein of any one of the above claims, wherein the protein is a dimer.

45. The chimeric protein of claim 44, wherein the protein is a non-covalently linked dimer

46. The chimeric protein of any one of the above claims, comprising the amino acid sequence of SEQ ID NO: 9, or a variant having at least about 90%, 95%, 97%, 98%, or 99% identity thereto.

47. A recombinant nucleic acid composition encoding one or more chimeric proteins of any one of the above claims.

48. A host cell comprising a nucleic acid of claim 47.

49. The chimeric protein of any one of claims 1-46, wherein the chimeric protein is suitable for use in a patient having one or more of: cancer, infections, immune disorders, autoimmune and/or neurodegenerative disease, cardiovascular diseases, wound, ischemia-related diseases, and/or metabolic diseases.

50. A method for treating or preventing a cancer, comprising administering an effective amount of the chimeric protein of any of claims 1-46 to a patient in need thereof.

51. The method of claim 50, wherein the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma;
oral cavity cancer (lip, tongue, mouth, and pharynx); ovarian cancer; pancreatic cancer; prostate cancer;
retinoblastoma; rhabdomyosarcoma; rectal cancer; cancer of the respiratory system; salivary gland carcinoma;
sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL;
mantle cell lymphoma; AIDS-related lymphoma; and Waldenstrom's Macroglobulinemia; chronic lymphocytic leukemia (CLL); acute lymphoblastic leukemia (ALL); hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

52. The method of claim 51, wherein the cancer is acute myeloid leukemia (AML).

53. A method for treating or preventing an autoimmune and/or neurodegenerative disease, comprising administering an effective amount of the chimeric protein of any of claims 1-46 to a patient in need thereof.

54. The method of claim 53, wherein the autoimmune and/or neurodegenerative disease is selected from multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, myasthenia gravis, Reiter's syndrome, and Grave's disease.


SEQUENCE LISTING (39 sequences)

SEQ ID NO: 1 - protein, 235 aa, Artificial Sequence
  Synthetic Sequence
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 2 - protein, 158 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQP

SEQ ID NO: 3 - protein, 155 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTA

SEQ ID NO: 4 - protein, 134 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQP

SEQ ID NO: 5 - protein, 129 aa, Artificial Sequence
  Synthetic Sequence
CSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQC

SEQ ID NO: 6 - protein, 165 aa, Artificial Sequence
  Synthetic Sequence
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 7 - protein, 164 aa, Artificial Sequence
  Synthetic Sequence
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEWRAEIMRSFSLSTNLQESLRSKE

SEQ ID NO: 8 - protein, 399 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAVDGGSGGSGGSGGSGGSGGSRSGGSGGSGGSGGSGGSGGSGGSGGSGGSGGSGGSGGSGGSAAAMCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKELEGGSHHHHHHHHH

SEQ ID NO: 9 - protein, 390 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAVDGGSGGSGGSGGSGGSGGSRSGGSGGSGGSGGSGGSGGSGGSGGSGGSGGSGGSGGSGGSAAAMCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKELEGGS

SEQ ID NO: 10 - protein, 5 aa, Artificial Sequence
  Synthetic Sequence
GGGGS

SEQ ID NO: 11 - protein, 10 aa, Artificial Sequence
  Synthetic Sequence
GGGGSGGGGS

SEQ ID NO: 12 - protein, 15 aa, Artificial Sequence
  Synthetic Sequence
GGGGSGGGGSGGGGS

SEQ ID NO: 13 - protein, 20 aa, Artificial Sequence
  Synthetic Sequence
GGGGSGGGGSGGGGSGGGGS

SEQ ID NO: 14 - protein, 25 aa, Artificial Sequence
  Synthetic Sequence
GGGGSGGGGSGGGGSGGGGSGGGGS

SEQ ID NO: 15 - protein, 30 aa, Artificial Sequence
  Synthetic Sequence
GGGGSGGGGSGGGGSGGGGSGGGGSGGGGS

SEQ ID NO: 16 - protein, 35 aa, Artificial Sequence
  Synthetic Sequence
GGGGSGGGGSGGGGSGGGGSGGGGSGGGGSGGGGS

SEQ ID NO: 17 - protein, 40 aa, Artificial Sequence
  Synthetic Sequence
GGGGSGGGGSGGGGSGGGGSGGGGSGGGGSGGGGSGGGGS

SEQ ID NO: 18 - protein, 16 aa, Artificial Sequence
  Synthetic Sequence
GGSGGSGGGGSGGGGS

SEQ ID NO: 19 - protein, 8 aa, Artificial Sequence
  Synthetic Sequence
GGGGGGGG

SEQ ID NO: 20 - protein, 6 aa, Artificial Sequence
  Synthetic Sequence
GGGGGG

SEQ ID NO: 21 - protein, 5 aa, Artificial Sequence
  Synthetic Sequence
EAAAK

SEQ ID NO: 22 - protein, 10 aa, Artificial Sequence
  Synthetic Sequence
EAAAKEAAAK

SEQ ID NO: 23 - protein, 15 aa, Artificial Sequence
  Synthetic Sequence
EAAAKEAAAKEAAAK

SEQ ID NO: 24 - protein, 12 aa, Artificial Sequence
  Synthetic Sequence
AEAAAKEAAAKA

SEQ ID NO: 25 - protein, 17 aa, Artificial Sequence
  Synthetic Sequence
AEAAAKEAAAKEAAAKA

SEQ ID NO: 26 - protein, 22 aa, Artificial Sequence
  Synthetic Sequence
AEAAAKEAAAKEAAAKEAAAKA

SEQ ID NO: 27 - protein, 27 aa, Artificial Sequence
  Synthetic Sequence
AEAAAKEAAAKEAAAKEAAAKEAAAKA

SEQ ID NO: 28 - protein, 46 aa, Artificial Sequence
  Synthetic Sequence
AEAAAKEAAAKEAAAKEAAAKALEAEAAAKEAAAKEAAAKEAAAKA

SEQ ID NO: 29 - protein, 5 aa, Artificial Sequence
  Synthetic Sequence
PAPAP

SEQ ID NO: 30 - protein, 18 aa, Artificial Sequence
  Synthetic Sequence
KESGSVSSEQLAQFRSLD

SEQ ID NO: 31 - protein, 14 aa, Artificial Sequence
  Synthetic Sequence
EGKSSGSGSESKST

SEQ ID NO: 32 - protein, 12 aa, Artificial Sequence
  Synthetic Sequence
GSAGSAAGSGEF

SEQ ID NO: 33 - protein, 5 aa, Artificial Sequence
  Synthetic Sequence
GGGSE

SEQ ID NO: 34 - protein, 5 aa, Artificial Sequence
  Synthetic Sequence
GSESG

SEQ ID NO: 35 - protein, 5 aa, Artificial Sequence
  Synthetic Sequence
GSEGS

SEQ ID NO: 36 - protein, 35 aa, Artificial Sequence
  Synthetic Sequence
GEGGSGEGSSGEGSSSEGGGSEGGGSEGGGSEGGS

SEQ ID NO: 37 - skipped

SEQ ID NO: 38 - protein, 166 aa, Artificial Sequence
  Synthetic Sequence
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 39 - protein, 153 aa, Artificial Sequence
  Synthetic Sequence
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS